(19) United States
(12) Patent Application Publication
(10) Pub. No.: US 2025/0160450 A1
(43) Pub. Date: May 22, 2025

(54) Compression Garment for Heat and Cold Therapy

(71) Applicant: Taylor Bond, San Diego, CA (US)

(72) Inventor: Taylor Bond, San Diego, CA (US)

(21) Appl. No.: 18/957,500

(22) Filed: Nov. 22, 2024

Related U.S. Application Data
(60) Provisional application No. 63/601,817, filed on Nov. 22, 2023.

(51) Int. Cl. A41D 13/005; A41D 13/00; A61F 7/02

(52) U.S. Cl. CPC A41D 13/0058; A41D 13/0015; A61F 7/02; A61F 2007/0219; A61F 2007/0234; A61F 2007/0236

(57) ABSTRACT

A thermal therapy compression garment for providing heat or cold therapy to the body of a user wearing the garment is provided. The garment features a garment body formed of elastic material which has a plurality of thermal storage components coupled to the body. With the garment in an as-worn position on the body of a user, heat or cold stored in the thermal storage components is communicated to the body of the user.

This application claims priority to U.S. Provisional Patent Application Ser. No. 63/601,817 filed on Nov. 22, 2023.

### BACKGROUND OF THE INVENTION

### 1. Field of the Invention

The invention herein disclosed relates generally to treatment for the human body by communication to portions of the body of heat or cold. More particularly, it relates a garment formed of elastic material or compressive material which is coupled to thermal-absorbent material whereby a compressive fit of the garment communicates either hot or cold therapy to the user on a portion of the body.

### 2. Prior Art

### Background of the Invention

In modern sports therapy, an ice bath, or sometimes cold-water immersion therapy, is widely employed as a cryotherapy treatment in the training regimen which usually follows a period of intense exercise. During such therapy a substantial part of the body of the athlete may be immersed in a bath of ice or ice-water for a limited duration. Such cryotherapy treatments can be a very time consuming process. They are also a location-limited process in that the athlete must have such therapy available at a facility, such as one having a deep soaking tub and the ability to fill it with very cold water.

Another therapy popular with athletes and for laypersons having muscle or skeletal pain in general, is heat therapy or thermal therapy. Similar to the above noted cold therapy, during heat therapy heat packs are applied to one or large areas of the body. Conventionally, such heat therapy is applied to the muscles and skeletal areas of the body to aid in healing since heat communicated to an area of the body increases blood circulation in those areas to promote healing. Such heat therapy is also known to provide an analgesic effect to help reduce pain.

However, with both conventional cold and heat therapies, the person undergoing such a treatment is limited in where such occurs by the availability of ice cold water for cold therapy, and the availability of either chemical heat packs or electrically heated pads or packs for heat therapy. Further, the person must be substantially immobile for both types of treatment, such as in a large tub of chilled water or sitting with a heat pad.

With respect to the above, before explaining at least one preferred embodiment of the compression garment configured for the provision of thermal therapy and method herein, it is to be understood that the disclosed device and method herein is not limited in its application to the details of employment and to any arrangement of steps set forth in the following description or illustrated in the drawings. The various apparatus components and configurations and methods of employment thereof as herein disclosed, are capable of other embodiments, and of being practiced and carried out in various ways, all of which will be obvious to those skilled in the art subsequent to their review of this specification.

Also, it is to be understood that the phraseology and terminology employed herein are for the purpose of description for an understanding of the thermal therapy compression system herein and should not be regarded as limiting. As such, those skilled in the art will appreciate that the conception upon which this disclosure is based may readily be utilized as a basis for other thermal therapy compression garments. It is important, therefore, that the embodiments, objects and claims herein, be regarded as including such equivalent construction and methodology insofar as they do not depart from the spirit and scope of the present invention.

### SUMMARY OF THE INVENTION

Thermal therapy and cryotherapy involve the communication of heat or cold to areas of the body. Such temperature-based therapy may be communicated for example and in now way limiting, to joints to improve the symptoms of osteoarthritis. Conventionally, such heat therapy is accomplished with soaking tubs, packs, towels, wax, and other modes of heat and cold transmission. Heat therapy or thermal therapy is considered to work by improving circulation to the areas where heat is communicated, and by relaxing muscles. Cryotherapy or cold therapy is conventionally employed to numb the pain, decrease swelling, constrict blood vessels and to block nerve impulses to the joints, muscles, and portions of the body being treated.

With the system and apparatus herein disclosed, professional level thermal therapy and cryotherapy can be employed easily in rehabilitation programs such as at professional centers or more conveniently, at home.

In all modes of provision of thermal therapy or cryotherapy the system herein employs a compression garment which once it is operatively positioned on a user, will communicate a compressive force toward the body of the user. The garment may include both a shirt portion and pants portion which may be used individually or in combination. Each portion of the compression garment is configured with a plurality of individual thermal storing components positioned thereon. This plurality of thermal storage components is operatively coupled to the compression garment. By operative coupling or coupled to the compression garment herein is meant that each of the thermal storage components is fixedly positioned in a position upon the fabric forming the compressive garment whereby the compression imparted by the fabric of the garment toward the skin of the wearer, will hold each thermal storage component adjacent to the skin where the compression fabric contacts the skin, or in contact with the skin of the wearer where the compression fabric is situated on one side of the thermal storage components. This coupling may include adhesive, fasteners, mounts, forming the weave or knit to surround both sides of each thermal storage component, fusing or RF welding the thermal storage components to the compression fabric, or other equivalent means for holding each thermal storage component in a fixed location on the garment as would occur to those skilled in the art subsequent to reading this disclosure.

The compression fabric herein is formed of woven or knitted or similar textile material having elastic yarns or threads included in the weave or knit, whereby when the weave or knit yarns and elastic yarns are stretched to position the garment on the wearer, the compression fabric imparts a pressured contact against the skin of the wearer. For example and in no way limiting compressive fabric may include Spandex fabrics which are widely available and provide a compressive engagement of the garment formed thereof and biasing against the skin and body of the user, or Clima Cool fabrics manufactured by Adidas. Such elastic fabrics may also include other synthetic fibers like nylon, polyester, or a blend of these fibers which are wrapped around a rubber core to create the stretch functionality, or other similar technical fibers forming fabrics which are breathable to help wick away moisture and designed to keep the wearer in comfortable, when sitting or when exercising.

By compression garment herein is meant any garment formed to be worn by a user which has compression, stretching or elastic material included in the weave or knit or other textile configuration therein, whereby the formed garment will stretch when pulled onto the body of the user, and thereafter will compress and impart a biasing force toward the skin and the body of the user when worn.

In the compression garment device herein and for the method herein, there are operatively coupled a plurality of thermal storage components. The thermal storage components are coupled to the fabric forming the compression garment in a number and size thereof configured to cover an area of the garment intended to impart heat or cold to a particular area of the body of a user when worn. By thermal storage component herein is meant any component which may be coupled to the elastic fabric forming a body, which will act as a thermal reservoir and store heat when heated and will store cold temperature when cooled. Such thermal storage components are widely available for example in a gel and the gel may be a solid gel or a liquid gel within flexible container, and combination gel configurations and/or other materials such as ceramic components, polymeric materials, water within containers, and other material or materials which are adapted to heat by heating in a heated compartment or cooled in a cooling compartment.

When these thermal storage components are operatively coupled upon an area of the compression fabric forming the compression garment, they will continuously communicate heat or cold as the case may be to the area of the body adjacent thereto. This heat or cold transfer works especially well when communicated against the body of the user when compressed toward the body by the compression garment they occupy.

Currently, such heat and cold absorbing and storage materials employable herein are formed in a wide variety of materials. Heat and cold absorbing gels are widely used and may be formed as a liquid within a sealed container, or may be formed to be stable and to be very soft or may be thickened to form pliable or soft but stable shaped gummy type components. For example only and in no way limiting, such gels include propylene glycol, ethylene or diethylene glycol. Many thermal storing materials contain some combination of hydroxyethyl cellulose for thickening, sodium polyacrylate, or silica gel.

As used herein, in addition to the above, thermal storage component means any heat or cold storage component formed of a heat sink and/or cold storing material, which will absorb and store heat or cold from a respective source thereof, and subsequently radiate it or communicate such to the body of a user wearing the compression garment bearing such thermal storage components.

The compression garment herein disclosed has a large plurality or number of such thermal storage components operatively coupled thereon. It has been found in experimentation, by coupling a large number of such thermal storage components to the elastic fabric material forming the compression garment, that heat or cold emanating therefrom is better communicated to the body of the user. This is because the perimeter of each such thermal storage component may be surrounded by an area of compression fabric material forming the compression garment and it biases the thermal storage components toward the skin and body of the user individually. Thus each individual thermal storage component is biased or compressed toward or against the body of the user by the stretched compression material surrounding the respective perimeter thereof.

A spacing of the individual thermal storage components from the others can be adjusted by forming them larger or smaller and/or by allowing for larger or smaller gaps formed by the surrounding perimeters of compressive fabric of the compression garment. This sizing and spacing option allows for the customization of the compression garment with more or less thermal transmission from the individual thermal storage components to thereby communicate more or less heat or cold to the user depending on the configuration.

Further, the shape of each individual thermal storage component may be configured to provide more or less thermal transmission to the body of the user wearing the compression garment on which they are mounted. For example and in no way limiting, round shaped thermal storage components may allow for more elastic fabric in-between each thermal storage component while triangular or rectangular shaped thermal storage components can occupy a much larger area of the elastic fabric to which they are coupled and thereby provide an increase in hot or cold thermal transmission to the body of the user. The shape of the thermal components may be any shape which provides the desired spacing between each thermal storage component and the flexibility of the garment when worn. In addition to circular and rectangular the shape may be any polygon shape or any shape having a curves in the perimeter edges.

The garment herein may include compressive garments forming shirts and/or pants or may be formed in a single unit of shirt and pants. The garment device may also include smaller sections such as short or long sleeves or leg portions for smaller or larger areas of treatment. A user wearing the compressive garment herein thus can employ it to provide cold treatments to large or small targeted areas of the body, without the need to sit in a tub as is the current mode of such treatment. The same convenience is provided for heat treatment to body areas as the user may simply wear the garment configured to provide heat to the area of the body requiring it for treatment.

In a method of employment of the garment herein, the area of the body of the wearer needing thermal treatment will be ascertained. The use of heat or cold will also be ascertained. Thereafter a garment having thermal storage components coupled to the area of the garment which will be positioned adjacent to the area of the body needing treatment will be chosen. The chosen garment may be taken from a heated or cold storage housing if they are pre heated or cooled, or may be heated or cooled as necessary for the treatment. Thereafter the garment will be placed in a compressive fit upon the body of the user for a duration of time determined to be appropriate for the treatment whereafter it can be removed.

With respect to the above summary description, before explaining at least one preferred embodiment of the thermal therapy compression garment device and system herein, it is to be understood that the invention is not limited in its application to the details of operation nor the arrangement of the individual components or the steps set forth in the following description or illustrations in the drawings. The various methods of implementation and operation of the thermal therapy compression garment device and system and method herein, are capable of other embodiments and of being practiced and carried out in various ways which will be obvious to those skilled in the art upon their review this disclosure. Also, it is to be understood that the phraseology and terminology employed herein are for the purpose of description and should not be regarded as limiting.

Therefore, those skilled in the art will appreciate that the conception upon which this disclosure is based may readily be utilized as a basis by others for designing of other modes for carrying out the several purposes of the present thermal therapy compression garment device and system. Therefore, the objects and claims herein should be regarded as including such equivalent construction, steps, and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. Where used herein, if not otherwise defined, the term “substantially” means plus or minus five percent.

It is an object of this invention to provide a compression garment for the imparting of therapeutic compression therapy to the wearer.

It is a further object of this invention to provide such a compression garment which includes a plurality of thermal storage components coupled thereon where each may have a compressed positioning being biased or urged toward or against the body of the user to provide heat or cold therapeutic therapy.

These together with other objects and advantages, which become subsequently apparent, reside in the details of the construction and operation of the disclosed thermal therapy compression garment device and system herein as more fully hereinafter described and claimed, reference being had to the accompanying drawings forming a part thereof, wherein like numerals refer to like parts throughout.

Further objectives of this invention may be ascertained by those skilled in the art as brought out in the following part of the specification wherein detailed description is for the purpose of fully disclosing the invention without placing any limitations thereon.

### BRIEF DESCRIPTION OF DRAWING FIGURES

The accompanying drawings, which are incorporated herein and form a part of the specification, illustrate some but not the only or exclusive examples of embodiments and/or features of the disclosed thermal therapy garment device and method herein. It is intended that the embodiments and figures disclosed herein are to be considered illustrative of the invention herein, rather than limiting in any fashion.

In the drawings:

FIG. 1 shows an exploded view of a shirt and pants forming a compression garment herein having a plurality of thermal storage components operatively coupled thereon.

FIG. 2 shows an example of an enlarged view of a shirt type of compression garment formed of a weave, knit, or other textile configuration having an elastic material included therein.

FIG. 3 depicts an enlarged view of a plurality or round shaped thermal storage components operatively coupled to the fabric forming the compression garment where the fabric as shown surrounds the perimeter of each.

FIG. 4 is a sectional view through elastic fabric material forming a compression garment herein showing the plurality of individual thermal storage components sandwiched between two layers of the woven, knit, or other textile fabric.

FIG. 5 depicts a mode of the device wherein the thermal storage components are rectangular.

FIG. 6 shows a mode of the device herein showing a section of the elastic material forming the compression garment wherein the thermal storage components are polygons such as triangles.

FIG. 7 depicts a thermal container into which the compression garment herein may be stored once the thermal storage components thereon have reached the desired hot or cold temperature so the device can be prepared in advance.

FIG. 8 shows a sectional view of the thermal storage components formed with planar sheets of thermal material on opposing sides of the individual components coupled to the elastic material forming the garment.

FIG. 9 shows a mode of the device herein wherein the individual thermal components have a planar sheet of thermal material to which they are formed which is coupled to the elastic material.

FIG. 10 depicts the thermal components positioned within individual pockets formed of and part of the elastic material forming the garment or engaged therewith.

FIG. 11 shows a configuration similar to that of FIG. 10 wherein each of the thermal components is positioned within a pocket formed by the elastic material forming the garment or engaged therewith.

FIG. 12 shows a configuration of the garment wherein seams are separable using magnetic coupling or hook and loop fabrics to allow all or portions of the front of the garment and front of the sleeve to be separated and reattached.

FIG. 13 shows a short sleeve configuration of the garment shirt wherein separable fasteners such as magnetic coupling or hook and loop fabric or other fasteners allow for the separation and re engagement of the front to the rear.

FIG. 14 shows an exploded view of at thermal housing adapted to generate heat or cold and communicate such to a garment positioned within a recess.

FIG. 15 shows the thermal housing of FIG. 14 assembled.

### DETAILED DESCRIPTION OF THE PREFERRED EMBODIMENTS OF THE INVENTION

In this description, the directional prepositions of up, upwardly, down, downwardly, front, back, top, upper, bottom, lower, left, right, first, second, and other such terms refer to the thermal therapy compression garment herein and components thereof as they are oriented and appear in the drawings and all such terms are used for convenience only and such are not intended to be limiting or to imply that the compression garment device and system have to be used or positioned in any particular orientation.

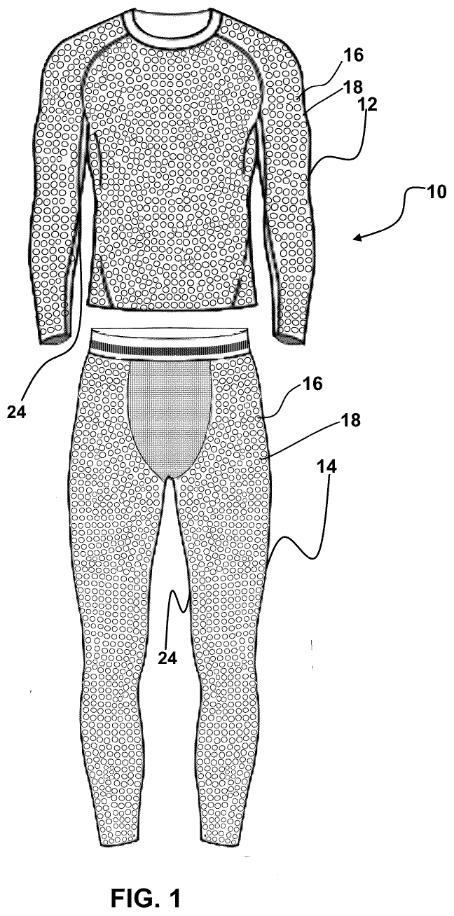

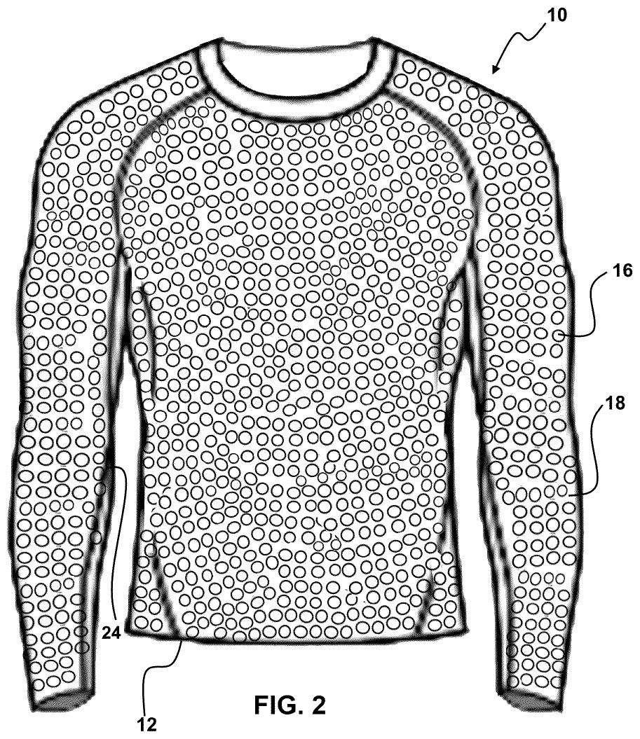

Now referring to drawings in FIGS. 1-15, there is seen, in FIGS. 1-2 depictions of the thermal therapy compression garment device 10 herein. As shown, the thermal therapy garment device 10 may include a garment body formed of elastic fabric 18 and the garment body may have a shirt portion 12 of the compression garment and/or a pants portion 14 thereof, which is formed of a woven or knitted or other textile fabric which is configured with yarns or other materials which stretch and impart compressive force or bias against the wearer when in an as-worn position. Thermal storage components are shown covering substantially an entire area of the garment body of elastic fabric 18. By substantially an entire areas is meant that the thermal storage components 16 are coupled in locations wherein but for the spacing between them and any perimeter edges, there are thermal storage components 16 covering the rest of the area. This positions elastic fabric 18 surrounding each thereby providing an even biasing force of each thermal storage component 16 in a direction toward the skin and body of the user.

Where the front and rear portions of the garment body of the garment device 10 are separable and re engageable, it may also be configured as a shirt portion 12, or a pants portion 14 or it may be formed as a “onesie” where the shirt portion 12 and pants portions 14 are formed in a single unit. Such elastic fabric preferably includes elastic thread or yarn such as spandex or Clima Cool fabric or other woven or knitted or formed textile fabric which will impart compression and biasing toward the skin of the body of the user.

Other smaller garment portions configured to engage a particular part or portion of the body of a user, while not shown, are considered within the scope of this application such as booties, gloves, collars and the like which have the individual thermal storage components upon elastic fabric 18 forming a body of the garment.

As shown in FIGS. 1-2, a plurality of thermal storage components 16 are operatively coupled to the elastic fabric 18 forming the a body of the garment device 10. The area of the garment portions covered by the thermal storage components 16 may be complementary to the area of the body of the user which is to be treated when the shirt portion 12 and/or pants portion 14 or other garment portion being worn by the user in an as-worn position. By as-worn position herein is meant the shirt portion 12 is situated on the body of the user with the arms and torso of the shirt covering the arms and torso of the user and/or the pants portion 14 is situated on the legs of the user and top end of the pants portion 14 covering below the torso. As noted, the garment device 10 herein, may be simply the shirt portion 12 or the pants portion 14 or both, or the garment may be provided as a single unit or onesie formed of the shirt portion 12 and pants portion 14. Of note, the sleeves on the shirt portion 12 and legs on the pants portion 14 may be full length or short.

As noted above the thermal storage components 16 are formed of a material which will provide a heat sink reservoir or a cold reservoir for use for the thermal therapy session as desired.

All portions of the body of the garment device 10 which have the thermal storage components 16 thereon, are preferably formed of elastic fabric 18 whether woven, knitted, or otherwise formed. In this fashion the elastic fabric 18 will first elongate and then will compress upon each individual thermal storage component 16 and bias or urge them in a direction toward or against the body of the user thereby providing better thermal transmission.

As noted, by surrounding each of the thermal storage components 16 with a perimeter of the elastic fabric 18, gaps are preferably formed between each thermo storage component 16 and each is thereby individually biased toward the skin of the user or wearer and the garment device 10 better forms to the contours of the body of the user. The biased positioning of the thermal storage components as noted better communicates heat or cold to portions of the body of the user, such as in those contours.

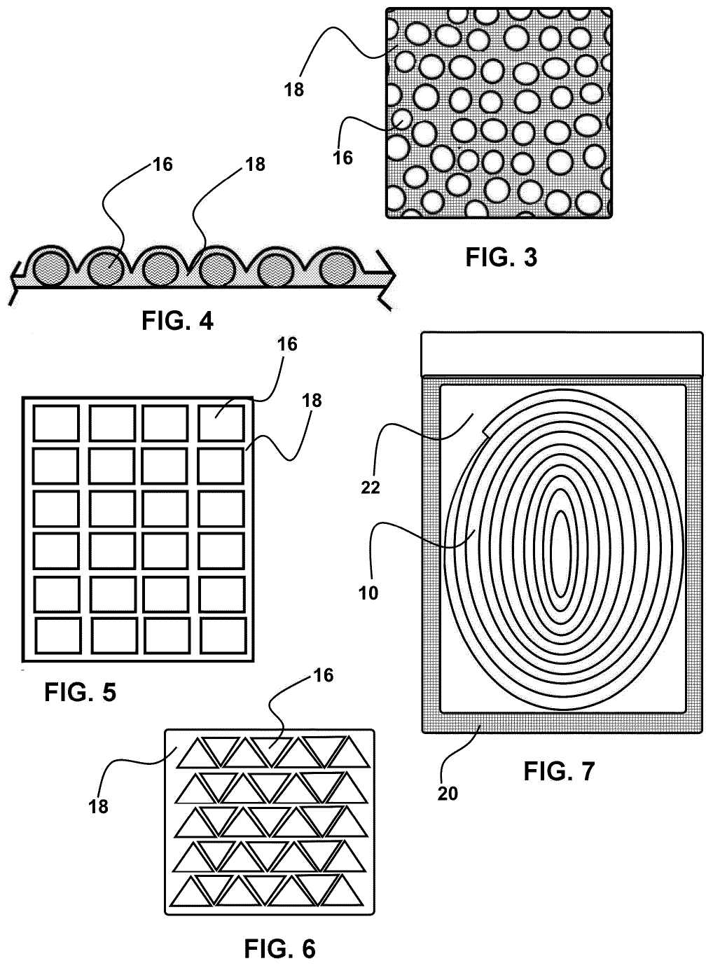

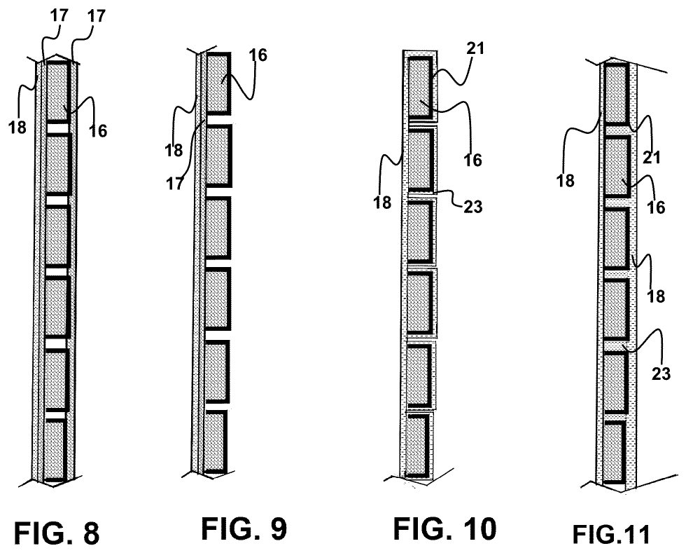

Shown in FIG. 3, is an enlarged area of the elastic fabric 18 forming a body of a garment device 10 showing the individual thermal storage components 16 operatively coupled to positions upon the elastic fabric 18. As noted, by operatively coupled herein is means that such engagement may be by adhesive, sonic welding, sewing, connectors, pockets or sleeves or openings in the fabric, or fabric surrounding both sides of the thermal storage components 16 or any manner where each of the thermal storage components 16 is secured to a substantially fixed position on or with the elastic fabric 18. Such engagement may also include forming multiple layers of the elastic fabric 18 such as in FIG. 4 and FIG. 8 and placing the thermal storage components 16 therebetween where they may be held by adhesive or sewing or other means as would occur to those skilled in the art. Other modes of engagement are shown in FIGS. 9-11.

As noted, the number and shape of each of the thermal storage components 16 may vary to allow more or less communication of heat or cold therefrom to the body of the user. As shown in FIG. 5, polygons such as those with rectangular shapes of the thermal storage components 16 allows for a tighter grouping upon the elastic fabric 18 and increased number of thermal components 16 as opposed to the round in FIG. 3. This may provide for increased communication of heat or cold therefrom to the body of the wearer when they are biased toward or against the body of the user.

FIG. 6 shows another mode of polygons for the thermal storage components 16. As shown they are formed in a triangular shape and coupled to the elastic fabric 18 of the garment device 10. This triangular shape has been shown in experimentation to allow for extra bending in along both angular and parallel sides of the thermal storage components 16 and to provide for an easier conforming of the areas of the garment device 10 herein which must conform to body contours and would be preferred for such. Additionally, the triangular shape may allow for a higher number of the thermal storage components 16 on a given garment device 10 and thereby allow for longer durations of heat or cold therapy.

Because the garment device 10 herein can be preheated or pre refrigerated, to impart heat or cold to the reservoirs for such provided by the thermal storage components 16, the garment device 10 allows it to be prepared for each user in advance. For athletes desiring cold therapy after a game or event, the garment device 10 may be held in a freezer or other cold storage to thereby chill the plurality of thermal storage components 16.

Where the user desires heat therapy to all or a portion of their body to be covered by a garment device 10 or portion thereof, the garment device 10 may be heated such as by a microwave oven, or within a heated storage locker. Once removed and worn by the user heat is communicated to the portions of the body of the user where thermal storage components 16 are located on a complementary area of the garment device 10. In either fashion, once pre prepared with heat or cold, the garment device 10 may be stored in an insulated container 20. When use is desired the user will simply remove it from the container 20 and place it upon their body to form the compressive engagement of the plurality of thermal storage components 16 for the therapy desired. In one mode of the garment device 10 a thermal housing may be provided which is capable of generating heat or cold using electric powered thermoelectric chips which may be operated to heat or cool the holding area of the thermal housing.

FIG. 7 depicts an example of a thermal container 20 into which the compression garment device 10 herein may be stored once the thermal storage components 16 thereon have reached the desired hot or cold temperature so the device can be prepared in advance. The thermal container 20 preferably has a storage recess 22 surrounded by insulated sidewalls to maintain the temperature of the thermal storage components 16.

Shown in FIG. 8 shows a sectional view of the thermal storage components 16 having planar sheets of thermal material 17 on opposing sides of the individual thermal components 16. Such may be formed in individual sections of thermal material 17 and individual thermal components 16 or as a unitary structure such as in a mold. The thermal storage components 16 and planar sheets of thermal material 17 are coupled to the elastic fabric 18 forming the garment device 10 and will be automatically positioned with spaces of both elastic fabric 18 surrounding the perimeter of each thermal storage component 16 along with a portion of the planar thermal material.

Another sectional view is shown in FIG. 9 the individual thermal storage components 16 have a planar sheet of thermal material 17 to which they are coupled or preferably formed in a unitary structure. As shown, the planar thermal material 17 on one side of each of the thermal storage components 16 is coupled to the elastic material 18. The planar thermal material 17 and the thermal storage components 16 are both formed of elastic or stretchable material such as a gel.

FIG. 10 depicts the thermal storage components 16 coupled to the elastic fabric 18 by the employment of individual pockets 21 formed as part of the elastic material 18 forming the garment device 10 or engaged therewith. Each pocket 21 holds an individual thermal storage component 16 in the spaced relationship noted above wherein it is surrounded by elastic material 18 to impart the bias of the thermal storage component 16 toward the skin of the user. Each of the pockets 21 as shown has separated sidewalls between the next pocket 21.

Depicted in FIG. 11 is a configuration similar to that of FIG. 10 wherein each of the thermal storage components 16 is positioned within a pocket 21. The pockets 21 may be formed by the elastic material 18 forming the garment or engaged therewith. In this configuration the pockets 21 have common sidewalls 23 between each adjacent pocket 21. This configuration and that of FIG. 10 may be formed during weaving or knitting of the elastic fabric 18 by insertion of a thermal storage component 16 into each pocket 21 thereby yielding sheets of the elastic fabric 18 with the thermal storage components 16 therein and properly spaced. The garment device 10 can then be cut from sheets of the formed fabric which may be sewn, fused, RF welded, or otherwise engaged along seams.

FIG. 12 shows a configuration of the shirt portions of the garment device 10 herein. As shown seams 24 which engage portions of the garment device 10 to each other such as a front half to a rear half, are separable and reattachable. This is accomplished using separable fasteners to hold the seams 24 together. Such separable fasteners may include for example magnetic coupling 26 where a strip of magnetic material on one half of the seam 24 is removably coupled with magnetically attractive material on the other side. While not shown, the same separable fastening of the seams forming the front and rear of the pants portion 14 may be used to allow users to separate and engage the seams when placing the garment device 10 upon their body.

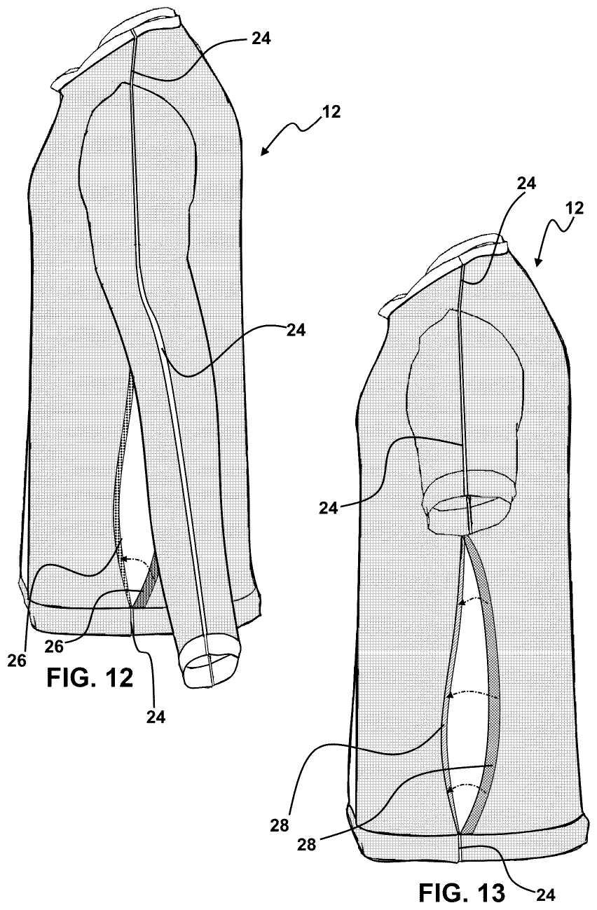

Another configuration of the garment device 10 formed of separable portions is shown in FIG. 13 which shows a short sleeve configuration of the shirt portion 12 wherein separable fasteners such as hook and loop fabric 28 are positioned on opposing sides of the same 24 engaging the front of the shirt to the rear thereof. As noted in the description above, while not shown, the same separable fastening of the seams 24 forming the front and rear of the pants portion 14 may be used to allow users to separate and engage the seams 24 when placing the pants portion of the garment device 10 upon their body.

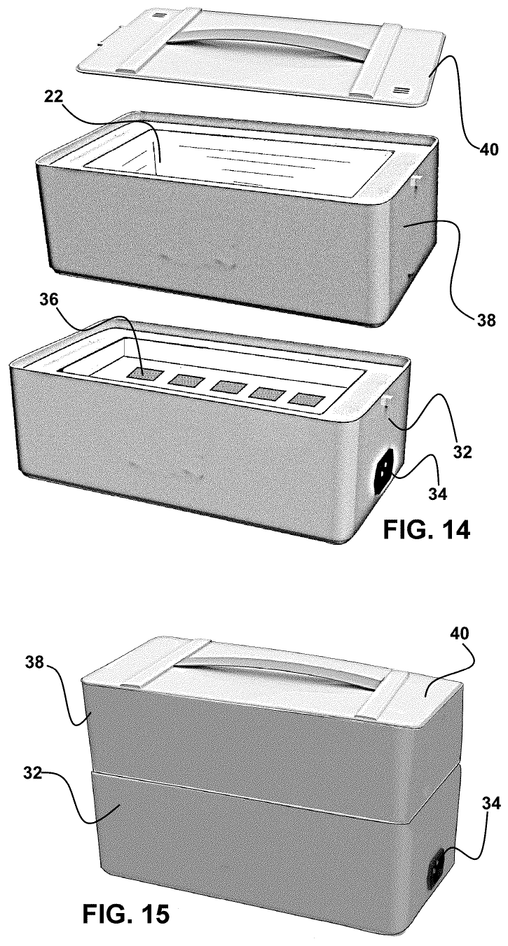

Shown in FIGS. 14-14 are a thermal container 20 which may be employed in combination with the garment device 10 herein. As shown, a base 32 has an electric connection 34 which may be engaged with an electric power source to energize thermoelectric chips 36 to either cool, or heat the storage recess 22 located in a secondary portion 38 of the thermal container 20. A lid 40 is employable to cover the storage recess 22. While shown as multiple components the thermal container 20 may be a unitary structure or single unit.

In a method of employment of the garment such as in FIGS. 1-2 and herein, in a first step area of the body of the wearer needing thermal treatment will be ascertained. In a next step for the area of the body of the wearer determined, a use of heat or cold therapy for that area will be ascertained. In another step a garment having thermal storage components coupled to the area of the garment, which will be positioned adjacent to the area of the body determined as needing treatment will be chosen. The garment chosen may have a shirt portion 12 and/or a pants portion 14.

Next, the chosen garment may be taken from a heated or cold storage housing if they are pre heated or cooled, or may be heated or cooled as necessary for the treatment. In a next step the chosen garment or components thereof will be placed in a compressive fit upon the body of the user for a duration of time determined to be appropriate for the treatment. In a final step the chosen garments so placed on the body off the user are removed.

While all of the fundamental characteristics and features of the disclosed compression garment for thermal therapy herein have been shown herein, with reference to particular embodiments thereof, a latitude of modification, various changes and substitutions are intended in the foregoing disclosure. It will be apparent that in some instances, some features of the thermal therapy garment may be employed without a corresponding use of other features without departing from the scope of the invention as set forth.

It should also be understood that various substitutions, modifications, and variations may be made by those skilled in the art, upon viewing this disclosure without departing from the spirit or scope of the invention. Consequently, all such modifications and variations and substitutions are considered included within the scope of the invention as defined by the following claims.

What is claimed is:

1. A thermal therapy compression garment comprising:
a garment body formed of elastic material;
a plurality of thermal storage components coupled to said body;
said thermal storage components for communicating stored heat or stored cold therefrom, to a body of a user wearing said garment in an as-worn position, whereby an area of a said body of said user may be treated with said heat or said cold by said thermal storage components coupled upon said garment body in a location complementary to said area of said body of said user.

2. The thermal therapy compression garment of claim 1 wherein each of said thermal storage components is surrounded by an area of said elastic material; and
said elastic material imparting a bias of said thermal storage components in a direct toward the skin of said user wearing said garment in said as-worn position.

3. The thermal therapy compression garment of claim 1 wherein said garment body includes a shirt portion having a shape of a shirt with sleeves.

4. The thermal therapy compression garment of claim 2 wherein said garment body includes a shirt portion having a shape of a shirt with sleeves.

5. The thermal therapy compression garment of claim 1 wherein said garment body includes a pants portion having a shape of a pair of pants.

6. The thermal therapy compression garment of claim 2 wherein said garment body includes a pants portion having a shape of a pair of pants.

7. The thermal therapy compression garment of claim 3 wherein said garment body includes a pants portion having a shape of a pair of pants.

8. The thermal therapy compression garment of claim 4 wherein said garment body includes a pants portion having a shape of a pair of pants.

9. The thermal therapy compression garment of claim 2 wherein said thermal storage components have a circular shape.

10. The thermal therapy compression garment of claim 2 wherein said thermal storage components have a polygon shape.

11. The thermal therapy compression garment of claim 9 wherein said thermal components are formed of a gel.

12. The thermal therapy compression garment of claim 9 wherein said thermal components are formed of a gel.

13. The thermal therapy compression garment of claim 9 wherein said plurality of thermal components cover substantially an entire area of said garment body.

14. The thermal therapy compression garment of claim 10 wherein said plurality of thermal components cover substantially an entire area of said garment body.

15. The thermal therapy compression garment of claim 11 wherein said plurality of thermal components cover substantially an entire area of said garment body.

16. The thermal therapy compression garment of claim 12 wherein said plurality of thermal components cover substantially an entire area of said garment body.
